(19) United States
(12) Patent Application Publication
(10) Pub. No.: US 2016/0354495 A1
(43) Pub. Date: Dec. 8, 2016

(54) Multicore Magnetic Particles

(71) Applicant: ENDOMAGNETICS LTD, Cambridge (GB)

(72) Inventors: Quentin John Harmer, Cambridge (GB); Eric Mayes, Cambridge (GB)

(73) Assignee: ENDOMAGNETICS LTD, Cambridge (GB)

(21) Appl. No.: 15/171,383

(22) Filed: Jun. 2, 2016

Related U.S. Application Data
(60) Provisional application No. 62/169,799, filed on Jun. 2, 2015.

(51) Int. Cl. A61K 49/18; A61K 41/00; A61K 49/06

(52) U.S. Cl. CPC A61K 49/1863; A61K 49/06; A61K 41/0052

(57) ABSTRACT

A multicore magnetic particle. In one embodiment, the magnetic particle includes a plurality of superparmagnetic cores embedded in a non-magnetic matrix. In another embodiment, the effective anisotropy energy barrier of the particle is larger than the sum of the anisotropy energy barriers of the individual superparamagnetic cores. In yet another embodiment, the superparamagnetic cores are close enough to interact magnetically by exchange coupling and dipole interaction. In still yet another embodiment, the specific loss power of the magnetic particle is greater than the specific loss power of an equivalent mass of individual superparamagnetic cores.

### RELATED APPLICATIONS

This application claims priority from U.S. Provisional Patent Application 62/169,799 filed on Jun. 2, 2015, the content of which is herein included by reference in its entirety.

### FIELD OF THE INVENTION

The invention relates generally to magnetic nanoparticles and more specifically to magnetic nanoparticles for use in biomedical imaging and magnetically-induced hyperthermia for treatment of disease.

### BACKGROUND OF THE INVENTION

The use of magnetic nanoparticles in medicine is well established. Magnetic nanoparticles are known for use as imaging contrast agents in Magnetic Resonance Imaging (MRI) and in both magnetic particle imaging (MPI) and photoacoustic imaging. Particles are also used for localising sentinel lymph nodes, iron replacement therapy, and magnetically-induced hyperthermia. The value of the particles in medicine relates both to their magnetic properties (e.g., influencing the relaxation of nearby water protons) and their size (e.g., being selectively filtered from lymph by lymph nodes, or being preferentially trapped in a tumor due to poor vasculature within the tumor). One promising application emerging for the therapeutic use of magnetic nanoparticles is magnetic hyperthermia.

By applying an alternating magnetic field to a location containing magnetic nanoparticles, the magnetic nanoparticles can be heated in vivo either to improve the response of diseased tissue to adjuvant therapies or to ablate such tissue. “Specific Absorption Rate” (SAR) is a figure of merit related to the thermal energy dissipated by a material used for magnetic hyperthermia. For magnetic nanoparticles, the SAR depends on many factors—the volume, the concentration of magnetic nanoparticles, the temperature, the magnetization and the anisotropy of the nanoparticles, and the frequency and amplitude of the applied magnetic field. SAR can be empirically calculated by multiplying the gradient of the heating curve of the material by the heat capacity (c) of the nanoparticle in a fluid. That is:

SAR=(ΔT/Δt)c

where SAR is measured in watts/kilogram (W/kg). All other factors being equal, particles can maximize their SAR when their volume is close to the transition between single magnetic domain particle and multidomain magnetic particle. Taking iron oxide as an example, the optimal particle diameter range for heating using magnetic hyperthermia is 20-100 nm.

However, if the particles are sufficiently large so as to maintain a magnetic domain, they can be attracted to one another, at high concentrations, through their dipole interactions. Under these conditions, the particles can create agglomerates of particles which results in their ceasing to function as individual particles. When this happens in a living being, there is a risk of creating an embolism. For example, iron oxide particles agglomerate when their diameter is greater than 20 nm, depending on the particle shape and temperature. Therefore, the single domain volume (and associated single domain diameter) should be viewed as an upper safety limit for the use of magnetic nanoparticles in vivo. Magnetic particles below the single domain size limit are termed ‘superparamagnetic’. The critical single domain diameter for iron and the critical single domain for iron oxides are described in the art and shown in Table 1.

TABLE 1

| | Single Domain Critical | Superparamagnetic |
|---|---|---|
| | Diameter (Study/author | Critical |
| Material | dependent) | Diameter |
| Metallic Iron | 17 nm-26 nm | 16 nm |
| Magnetite | 19 nm-128 nm | 21 nm |
| Maghemite | 26 nm-91 nm | 35 nm |

Making particles of an “optimum particle size” out of magnetite or maghemite is challenging because the manufacturing processes available will inevitably produce a distribution of particle sizes in the solution, resulting in a large number of particles which are above the critical superparamagnetic diameter. Furthermore, the larger the particles, the more difficult they are for the body to break down for elimination because of their larger mass and smaller surface area-to-mass ratio. Therefore, it is desirable to minimize the size of the constituent particles in order to facilitate dissolution in the body. Although the optimal particle diameter for heating is greater than 20 nm, the optimal diameter for superparamagnetic behaviour and safe in vivo use is less than 20 nm and preferably in the range 4-12 nm. In one embodiment, the size is 5 nm.

Superparamagnetic particles primarily generate heat when exposed to a magnetic field through magnetic spin (Néel) relaxation, and SAR mostly depends on their magnetic anisotropy and volume. The published literature shows that the heating effect from superparamagnetic iron oxide particles decreases significantly as the particle volume decreases. For example, a 5.3 nm diameter particle has a Specific Loss Power (SLP), a measure of the heating effect similar to SAR, of 4 W/g, while a particle of 16.5 nm diameter has a SLP of 1650 W/g.

Thus, while it is possible to heat tissue in the presence of small superparamagnetic nanoparticles, the efficiency is low and therefore higher magnetic fields (or frequencies) are needed to achieve the same heating. However, the higher field strengths and field frequencies risk exposing the patient to unwanted tissue heating caused by eddy currents generated in healthy tissue. Brezovich showed empirically that if the product of f*H (frequency times magnetic field strength) is of the order of 4.85×10⁸, the subject will begin to feel warm but not uncomfortable after one hour of being illuminated by magnetic fields at that level. Thus, the f*H product needs to be kept below this value to avoid damage to, or unwanted effects in, healthy tissue. This means that heating with small iron oxide particles is inefficient and not clinically practical because the field strength and frequency combination needed to heat the particles to a therapeutic temperature will cause unwanted effects in healthy tissue.

With an upper safety limit to avoid agglomerates established for particle volume, the anisotropy energy barrier (the energy needed to reverse the magnetization direction of the magnetic domain) is generally the single parameter available for manipulation to optimize SAR for a given nanoparticle material and given applied magnetic field frequency. Although materials with magnetocrystalline anisotropies greater than that of iron oxide are available, only iron oxides have demonstrated good tolerability in the body. Alternative shapes such as cubic nanoparticles can increase the anisotropy and SAR compared with spherical particles of similar size, however fabrication of such cubic nanoparticles can be challenging. If interactions between particles are considered, the overall energy barrier term increases to include the anisotropy energy barrier alongside interparticle interactions such as magnetic dipole-dipole and exchange coupling.

Recent efforts have shown it may be possible to increase SAR by an order of magnitude through the formation of core-shell nanoparticles. By layering hard and soft magnetic layers within the nanoparticle structure, interfacial exchange interactions between the core and the shell (or the hard and soft magnetic layers) increase the overall energy barrier. However, the structures demonstrated to date utilize materials that are not well tolerated, which is of particular concern as nanoparticles may aggregate, generate harmful metabolites, and redistribute to vital organs within the body.

Thus, there exists a need for a magnetic hyperthermia system with particles that have a high SAR for efficient heating to minimize magnetic field exposure and unwanted eddy current heating, but are small enough to maintain superparamagnetic behavior so that they: a) do not agglomerate; b) are broken down by the body; and c) are well tolerated by the body.

The present invention addresses this need.

### SUMMARY OF THE INVENTION

In one aspect, the invention relates to a multicore magnetic particle. In one embodiment, the magnetic particle includes a plurality of superparmagnetic cores embedded in a non-magnetic matrix. In another embodiment, the effective anisotropy energy barrier of the multicore particle is larger than the sum of the anisotropy energy barriers of the individual superparamagnetic cores. In yet another embodiment, the superparamagnetic cores are close enough to interact magnetically by exchange coupling and dipole interaction. In still yet another embodiment, the specific loss power of the magnetic particle is greater than the specific loss power of an equivalent mass of individual superparamagnetic cores.

In one embodiment, the spacing between the superparamagnetic cores is less than 10 nm. In another embodiment, the spacing between the superparmagnetic cores is less than 5 nm. In still yet another embodiment, the spacing between the superparmagnetic cores is less than 2 nm.

In one embodiment, the superparamagnetic cores comprise maghemite. In another embodiment, the non-magnetic matrix is selected from the group consisting of carboxylic or hydroxycarboxylic acids, mono-, di- or polysaccharides, synthetic or biological polymers particularly hydrophilic polymers, acrylates, siloxanes, styrenes, acetates, akylene glycols, alkylenes, alkylene oxides, parylenes, lactic acid, and glycolic acid. In yet another embodiment, the non-magnetic matrix is selected from the group consisting of dextran and dextran derivatives, carboxydextran, heparin, heparin sulfate, polyacrylic acid, starch, silica, polyethyleneglycol (PEG), PEG-phosphatechitosan, glycosaminoglycan, cellulose, chondroitin sulfate, chitin, chitosan, dextrin, maltodextrin, polymaltose, xanthum gum, alginates, starches, and their derivatives, hydrophilic polymers or mixtures thereof. In still yet another embodiment, the non-magnetic matrix comprises carboxydextran.

### BRIEF DESCRIPTION OF THE DRAWINGS

The structure and function of the invention can be best understood from the description herein in conjunction with the accompanying figures. The figures are not necessarily to scale, emphasis instead generally being placed upon illustrative principles. The figures are to be considered illustrative in all aspects and are not intended to limit the invention, the scope of which is defined only by the claims.

FIG. 1A depicts an embodiment of a coated nanoparticle known to the prior art.

FIG. 1B depicts an embodiment of a multicore nanoparticle according to the present invention.

FIG. 2 depicts the features of an embodiment of a multicore particle according to the present invention.

FIG. 3 is a graph of the effect of core particle size on specific absorption rate of iron oxide particles.

FIG. 4 is a graph of temperature change with time for an embodiment of nanoparticles of the present invention.

FIG. 5 is a graph of the hydrodynamic particle size distribution of the particles of the present invention by Nanoparticle Tracking Analysis.

FIG. 6 is a graph of the heating of nanoparticles as known to the prior art.

### DESCRIPTION OF A PREFERRED EMBODIMENT

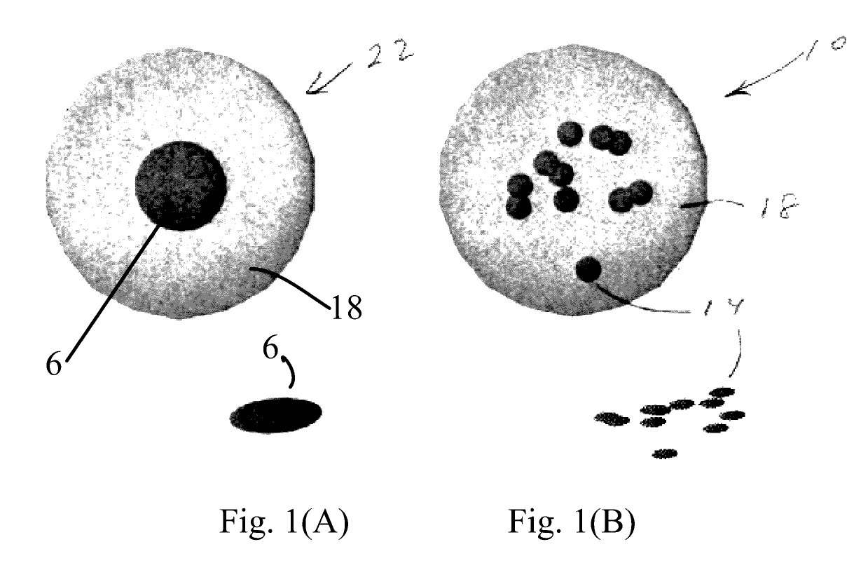

In brief overview and referring FIG. 1(A), a prior art single core nanoparticle 22 is shown, in which a single magnetic core 6 is located in a matrix 18. FIG. 1B, the present invention relates to a novel multicore nanoparticle 10 for use in magnetic hyperthermia therapy. In one embodiment, the multicore nanoparticle 10 of the present invention includes multiple superparamagnetic core particles (also referred to as cores) 14, based on well-tolerated iron oxide, in a non-magnetic matrix 18. Rather than core-shell interfacial exchange interactions, collective exchange and dipolar coupling between individual iron oxide cores increases the effective overall energy barrier, thereby increasing their effective volume. The proximity of the collection of smaller cores mimics a larger volume particle with a size close to the transition between single-domain and multi-domain particles. Further, with the nanoparticle cores being separated by a non-magnetic matrix, the matrix can be selected for enhanced degradation to minimize retention time in the body. Similar degradable bridges have been demonstrated in the delivery and clearance of non-magnetic composite inorganic nanoparticle assemblies. In addition, the non-magnetic matrix may be further functionalized to selectively target specific biomarkers or modify the external dimensions of the particle to enhance uptake in specific tissues.

By assembling multiple superparamagnetic cores 14 separated by a non-magnetic matrix 18 into a larger nanoparticle (FIG. 1B), the dynamic response of the particle can be equivalent to a single larger particle 22 (FIG. 1A) due to exchange and dipolar interactions between cores. Further, through selection of the material comprising the non-magnetic matrix, enhanced degradation of the nanoparticle can be engineered such that the particle clears more rapidly following the treatment sessions. The multicore particles help to maximize SAR using well-tolerated iron oxide without becoming ferromagnetic at room temperature. Moreover, selective cleaving of the connections between cores based on pH or an enzyme restriction site can increase the rate of nanoparticle degradation and clearance following use.

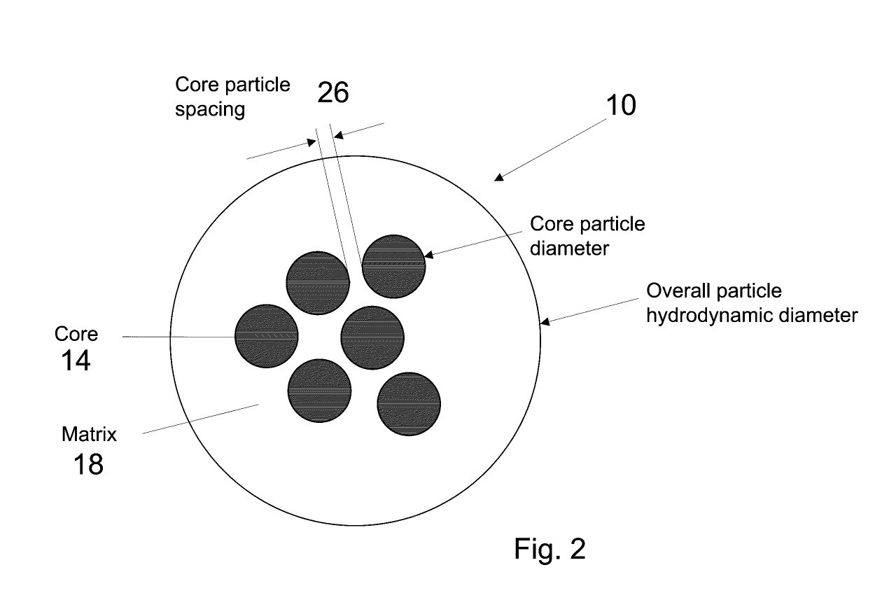

Referring to FIG. 2, in one embodiment, a multicore particle 10 of the present invention includes a plurality of cores 14 formed from a metal oxide, in one embodiment iron oxide. In another embodiment, the core material comprises the maghemite form of iron oxide. In one embodiment, the number of cores ranges from 2 to 30 in number, more preferably 5 to 15 in number. The “effective size” (the diameter of a single core particle that would give the same magnetic behavior) of the cluster of particles in the multicore particle is between 10 and 40 nm in diameter, more preferably between 20 and 30 nm in diameter. Thus, the multicluster cores behave magnetically in a similar way to a single particle of the same “effective size”.

The spacing between the cores 26 is chosen to facilitate dipole-dipole interactions and, potentially, exchange coupling between the core particles. For dipole-dipole interactions, the distance between the cores, in one embodiment, is less than around 10 nm, preferably less than 7 nm and more preferably less than 5 nm. For exchange coupling to occur in the multicore particle, the distance between the cores needs to be less than about 2 nm, preferably less than 1 nm, and more preferably in direct contact.

In one embodiment, the matrix 18 or coating around the cores 14 is formed from a biocompatible material that is well tolerated in the body. Preferably the material is biodegradable and readily broken down by the body into degradation products that can be readily absorbed or removed from the body. Suitable materials include carboxylic or hydroxycarboxylic acids, mono-, di- or polysaccharides, synthetic or biological polymers particularly hydrophilic polymers, acrylates, siloxanes, styrenes, acetates, akylene glycols, alkylenes, alkylene oxides, parylenes, lactic acid, and glycolic acid. Examples include dextran and dextran derivatives, carboxydextran, heparin, heparin sulfate, polyacrylic acid, starch, silica, polyethyleneglycol (PEG), PEG-phosphatechitosan, glycosaminoglycan, cellulose, chondroitin sulfate, chitin, chitosan, dextrin, maltodextrin, polymaltose, xanthum gum, alginates, starches, and their derivatives, hydrophilic polymers or mixtures of these. In one embodiment, the coating material is carboxydextran.

The processes for making such coated particles are well know to the art (see for example U.S. Pat. No. 5,055,288 and U.S. Pat. No. 5,7665,72). In one recipe, 161 g of a carboxydextran having an intrinsic viscosity of 0.050 dl/g is dissolved in 545 ml of water. An aqueous solution is obtained by dissolving, in a nitrogen environment, 22.3 g of ferrous chloride tetrahydrate in 228 ml of a 1M aqueous ferric chloride solution (corresponding to 61.6 g of ferric chloride hexahydrate). 520 ml of a 28% ammonia water is added in 30 seconds with heating at 80° C. and stirring. The resulting solution is then adjusted to pH 7.0 by adding 6N hydrochloric acid, after which the solution is heated and refluxed for 1 hour.

After cooling, the solution is centrifuged at 2,100 G for 30 minutes. An amount of ethanol is added equal to 78% of the supernatant volume to precipitate a complex. Centrifugation is conducted again at 2,100 G for 10 minutes and the resulting precipitate is dissolved in water and dialyzed against running water for 16 hours.

The dialyzate is adjusted to pH 7.2 using sodium hydroxide and concentrated under reduced pressure. The concentrate is filtered through a membrane filter (pores size: 0.2 μm) to obtain 92 ml of an intended complex aqueous solution having the following parameters: 137 mg/ml (iron yield: 66%), particle diameter of magnetic iron oxide: 6.6 nm, total particle diameter: 43 nm, water-soluble carboxypolysaccharide/iron weight ratio: 0.72, magnetization at 1 tesla: 88 emu/g of iron, T₂—relaxivity: 124 (mM. sec). The multicore particles are then separated using free-flow magnetophoresis to separate the particles by size and magnetic properties. Other separation techniques are available.

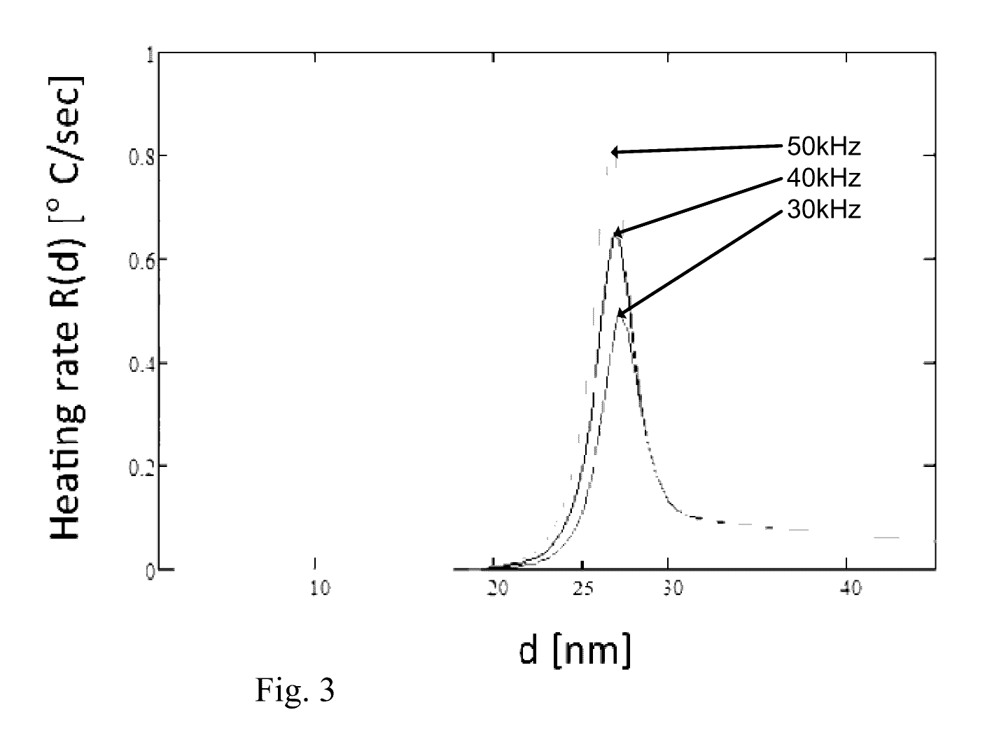

The effect of core size and magnetic field frequency on the heating rate of iron oxide particles is shown in FIG. 3. This graph was generated by modeling the magnetic response of single core iron oxide particles of different sizes at three different magnetic excitation frequencies. The peak for heating comes when the particle size is between around 25 and 30 nm and is negligible with a 5 nm core size.

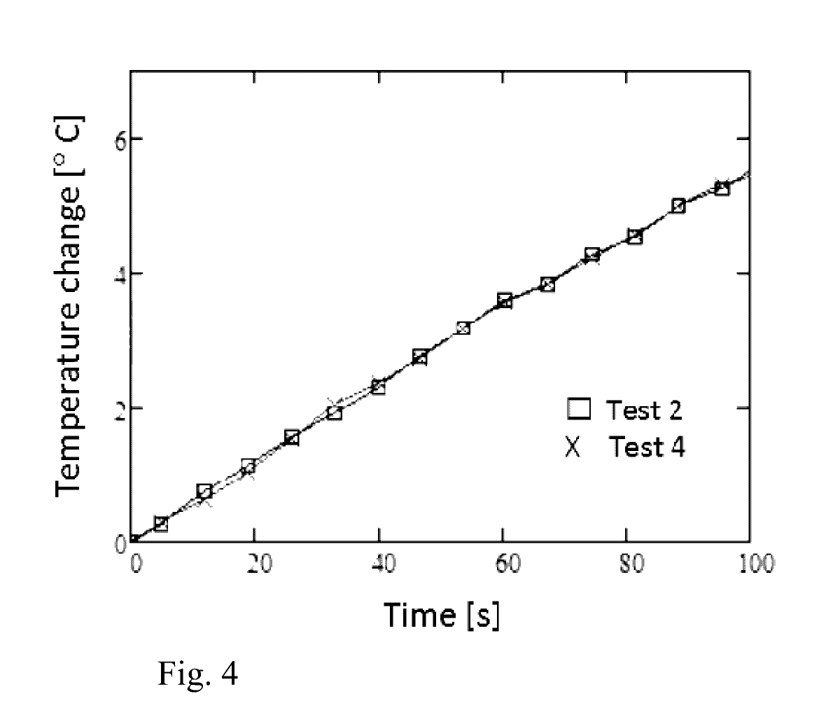

FIG. 4 is a representative graph of temperature versus time when the multicore particles of the present invention are heated. To generate this graph, an aqueous suspension of the multicore nanoparticles, comprising cores of iron oxide in a carboxydextran coating at a concentration of 28 mg/ml (volume fraction of iron oxide 0.78%), was placed in an alternating magnetic field of magnitude 5000 A/m at a frequency of 41 kHz. The heating rate is about 0.012° C./s.

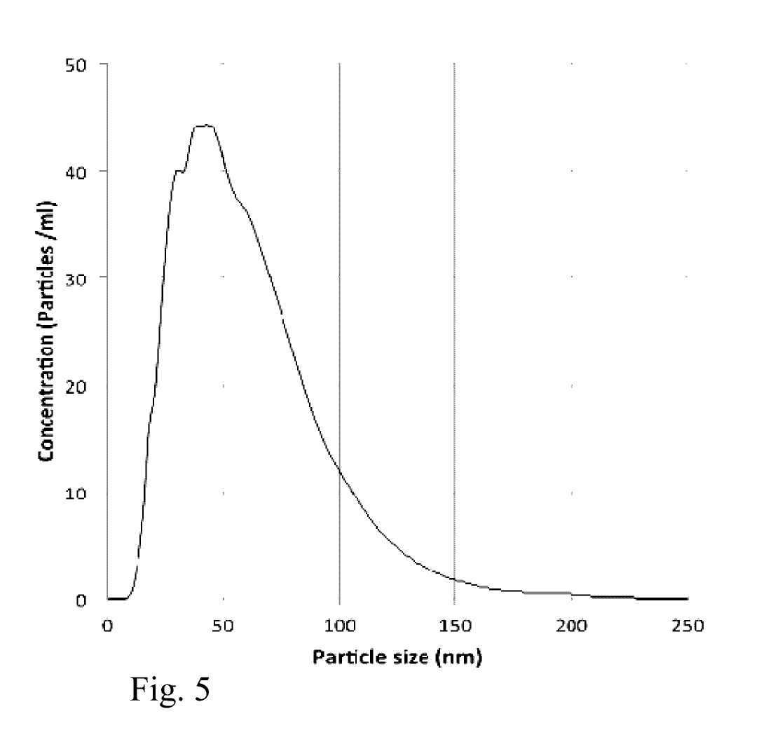

FIG. 5 is a graph of the hydrodynamic particle size distribution of the particles of the present invention. This graph illustrates the size of most of the carboxydextran-coated iron oxide particles being about 30-80 nm in diameter as measured by Nanoparticle Tracking Analysis. In this technique, an instrument (Nanosight Ltd, Amesbury, Wiltshire, UK) having an ultramicroscope with a video camera views a sample of nanoparticles illuminated by a laser as the particles move under Brownian motion. The motion of each nanoparticle is tracked by computer and the distribution of particle sizes is determined using the Stokes-Einstein equation.

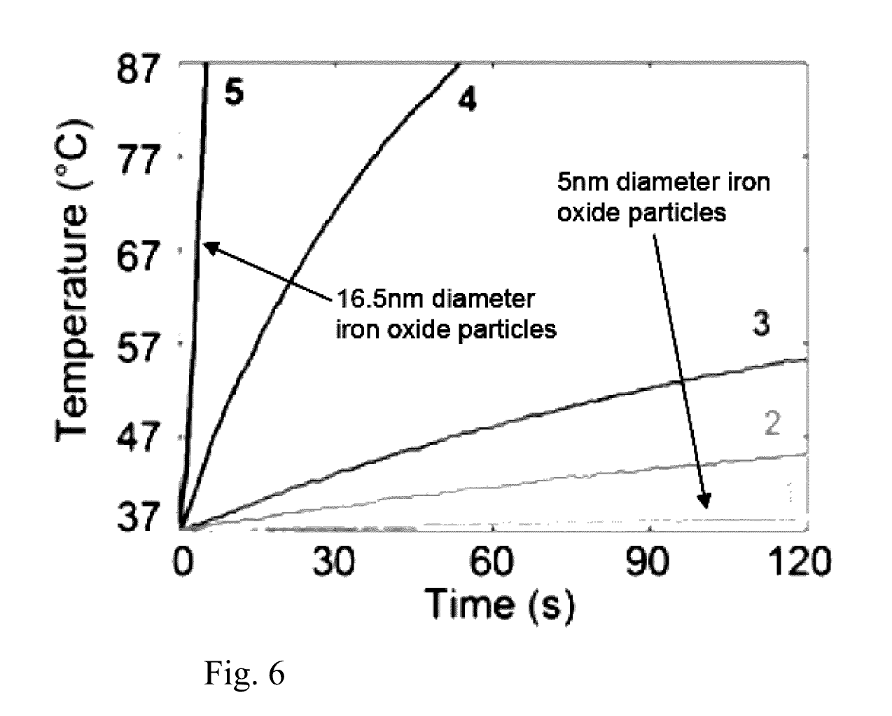

FIG. 6 is a graph of the heating results for single core particles as known to the prior art.

Table 2 is a comparison of the characteristics and heating effect of a prior art system using single core particles and the multi-core particles of the present invention. The single core measurements reflected in this table are from a prior art system with single core particles of 5 nm diameter positioned within a magnetic field strength of 24.8 kA/m, at a frequency of 700 kHz. For a single particle size of 5 nm, the heating rate is approximately 0.002° C./s. The multicore measurements used multicore particles also with a core size of 5 nm. In this system, the magnetic field magnitude was 5000 A/m and frequency is 41 kHz. As can be seen from this table, the heating rate is much higher at about 0.012° C./s.

TABLE 2

| | Prior art single | Present |
|---|---|---|
| | core particles | Invention |
| Core particle size (nm) | 5 | 5 |
| Magnetic field strength, H (A/m) | 24800 | 5000 |
| Frequency, f (kHz) | 700 | 41 |
| Relative fH² normalized to present | 419 | 1 |
| invention | | |
| Volume fraction of iron oxide (%) | 0.45 | 0.78 |
| Heating rate (deg C./s) | 0.002 | 0.012 |
| Relative heating rate | 1 | 6 |
| Relative tissue heating (fH, | 85 | 1 |
| normalized to present invention) | | |

In addition to SAR and SLP, the product f*H² is often used to compare the magnetic heating energy input between different systems. The table shows that the f*H² product is over 400 times higher for the prior art system, meaning that two orders of magnitude more energy is available to heat the particles. However, the heating effect in the prior art particles is relatively poor because particles of that size have a very low specific absorption rate (FIG. 3). Surprisingly, although their core particle size is similar, the heating properties of the multicore particles are significantly superior, even after allowing for the slightly more concentrated multicore particle solution used in this embodiment.

Further, because tissue heating is proportional to the product of frequency and magnetic field strength, f*H, the table shows that in order to deliver the heating effect of the prior art system of the table, 85 times more energy will be delivered to surrounding tissue than to achieve the heating shown using the multicore particles of the present invention. Thus, the particles of the present invention allow a far more efficient magnetic heating and consequently reduce the amount of unwanted tissue heating.

Unless otherwise indicated, all numbers expressing lengths, widths, depths, or other dimensions, and so forth used in the specification and claims are to be understood in all instances as indicating both the exact values as shown and as being modified by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques. Any specific value may vary by 20%.

The terms “a,” “an,” “the,” and similar referents used in the context of describing the invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claim. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified, thus fulfilling the written description of all Markush groups used in the appended claims.

Certain embodiments are described herein, including the best mode known to the inventor for carrying out the spirit of the present disclosure. Of course, variations on these described embodiments will become apparent to those of ordinary skill in the art upon reading the foregoing description. The inventor expects skilled artisans to employ such variations as appropriate, and the inventor intends for the invention to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

What is claimed is:

1. A multicore magnetic particle comprising:
a plurality of superparmagnetic cores embedded in a non-magnetic matrix,
wherein the effective anisotropy energy barrier of the multicore magnetic particle is larger than the sum of the anisotropy energy barriers of the individual superparamagnetic cores.

2. A magnetic particle comprising:
a plurality of superparmagnetic cores located in a non-magnetic matrix,
wherein the superparamagnetic cores are close enough to interact magnetically by exchange coupling and dipole interaction, and
wherein the specific loss power of the magnetic particle is greater than the specific loss power of an equivalent mass of individual superparamagnetic cores.

3. The magnetic particle of claim 1 wherein the spacing between the superparamagnetic cores is less than 10 nm.

4. The magnetic particle of claim 1 wherein the spacing between the superparmagnetic cores is less than 5 nm.

5. The magnetic particle of claim 1 wherein the spacing between the superparmagnetic cores is less than 2 nm.

6. The magnetic particle of claim 1 wherein the superparamagnetic cores comprise maghemite.

7. The magnetic particle of claim 1 wherein the matrix is selected from the group consisting of carboxylic or hydroxycarboxylic acids, mono-, di- or polysaccharides, synthetic or biological polymers particularly hydrophilic polymers, acrylates, siloxanes, styrenes, acetates, akylene glycols, alkylenes, alkylene oxides, parylenes, lactic acid, and glycolic acid.

8. The magnetic particle of claim 7 wherein the matrix is selected from the group consisting of dextran and dextran derivatives, carboxydextran, heparin, heparin sulfate, polyacrylic acid, starch, silica, polyethyleneglycol (PEG), PEG-phosphatechitosan, glycosaminoglycan, cellulose, chondroitin sulfate, chitin, chitosan, dextrin, maltodextrin, polymaltose, xanthum gum, alginates, starches, and their derivatives, hydrophilic polymers or mixtures thereof.

9. The magnetic particle of claim 1 wherein the non-magnetic matrix comprises carboxydextran.
